(19) United States
(12) Patent Application Publication
(10) Pub. No.: US 2004/0241064 A1
(43) Pub. Date: Dec. 2, 2004

(54) Portable photoelectric air cleaner

(75) Inventor: Se Kit Yuen, Hong Kong (CN)

Correspondence Address: SUGHRUE MION, PLLC, 2100 PENNSYLVANIA AVENUE, N.W., SUITE 800, WASHINGTON, DC, 20037, US

(73) Assignee: Se Kit YUEN

(21) Appl. No.: 10/698,441

(22) Filed: Nov. 3, 2003

(30) Foreign Application Priority Data
May 26, 2003 (CN) 03261667.8

(51) Int. Cl. B32B 5/02; A62B 7/08; B32B 27/04; B32B 27/12; A61L 2/00; A61L 9/00; B01J 19/08

(52) U.S. Cl. 422/292; 422/121; 422/300; 422/305; 422/306; 422/186; 422/124

(57) ABSTRACT

A portable photoelectric air cleaner, which includes a semi-circular body in which there are an extractor fan, a transformer, a circuit board, an electrode end ultraviolet radiation tube and a cathode high voltage discharge fibre line, which is characterized in that an air inlet and an outlet are formed on said body. The cathode fibre line for discharging in high voltage is fixed at the air outlet. The electrode end ultraviolet radiation tube is fixed at the outlet which is placed in the body, and is formed in an air collector. The extractor fan is fixed between the electrode end ultraviolet radiation tube and the air outlet which are formed nearly inside the body. The portable photoelectric air cleaner is furthermore characterized in that said air collector is comprised of a space which is defined by an air collecting wall and blocking wall. The electrode end ultraviolet radiation tube is fixed between the front and rear block walls so that the rays can't emit out of the cleaner.

### FIELD OF THE INVENTION

The present invention relates to a portable photoelectric air cleaner for killing the virus, bacterium, mildew etc in the air with ultraviolet and negative ions to clean the air, more particularly, to a device for generating ultraviolet and negative ions being powered by batteries and operated alternately in combination.

### BACKGROUND OF THE INVENTION

The providing of warm-air and cold-air in winter and summer causes the room air not to flow, so it is necessary to clean the air manually. Particularly, the people is paying more and more attention on the cleaning of the indoor air due to the diffusion of virus of SARS. For cleaning the room air, it is necessary not only to refresh the indoor air but also to kill the virus, bacterium, mildew etc in the air. Recently, the typical means of cleaning the air are air cleaners which generate the negative ions by ionizing in high voltage, some air-conditioners have such air cleaners. In addition, some ultra-violet sterilization lamps used in hospitals can kill the bacterium in the air effectively. Both of the above means for cleaning the air have their advantages, but there exists some obvious problems, for example, the effect of killing the bacterium with negative ions is not satisfied, the ultra-violet sterilization lamp can't be used for long time, the body will be hurt by irradiated rays. Furthermore, the typically air cleaners must be mounted immovably, or being used as a desk device in indoors, it is inconvenient for users to carry the cleaner, so we can't obtain the cleaned air when entering into the place not being provided with the air cleaner.

### Objects and Advantages

The object of the present invention is to overcome the problem in the prior art and provides a portable photoelectric air cleaner which cleans the air by combination of the ultraviolet and negative ions, so that the user can obtain the fresh and clean air everywhere at any time.

The object of the present invention is achieved by the following technical schemes. A portable photoelectric air cleaner, which includes a semi-circular body in which there are an extractor fan, a transformer, a circuit board, an electrode end ultraviolet radiation tube and a cathode high voltage discharge fibre line, which is characterized in that an air inlet and an outlet are formed on said body. The cathode fibre line for discharging in high voltage is fixed at the air outlet. The electrode end ultraviolet radiation tube is fixed at the outlet which is placed in the body, and is formed in an air collector. The extractor fan is fixed between the electrode end ultraviolet radiation tube and the air outlet which are formed nearly inside the body.

The portable photoelectric air cleaner is furthermore characterized in that said air collector is comprised of a space which is defined by an air collecting wall and blocking wall. The electrode end ultraviolet radiation tube is fixed between the front and rear block walls so that the rays can't emit out of the cleaner.

The portable photoelectric air cleaner is further characterized in that a supporting frame is formed in said body. The lower end of the supporting frame is provided with a power supply generator, a transformer, power source lines and a charging battery, and the upper end of the supporting frame is provided with electronic switcher.

The portable photoelectric air cleaner is further characterized in that a movable handle and a movable gallus are fixed on the body. Said movable gallus is connected to the body through a buckle.

The portable photoelectric air cleaner, is further characterized in that the body has charging batteries for providing power to the air cleaner, the output terminal of said charging batteries is connected to that of the direct current(DC) input of a circuit board.

The advantage of the portable photoelectric air cleaner according to the utility model is that: the negative ion is generated by making the carbide fibre line discharged and the ultraviolet radiation is emitted out by the electrode end ultraviolet radiation tube, thereby it can achieve the object of cleaning the air effectively and entirely through operating the above modes alternatively; and the design of the shape and configuration of the cleaner as well as the charging batteries which are formed inside thereof is provided to the users for being able to breathe the pure and fresh air which has been eliminated the virus, bacterium, mildew etc, which could prevent respiratory tract infection effectively.

### DESCRIPTION OF THE DRAWINGS

FIG. 1 is an outline side view of the product according to the present invention;

FIG. 2 is a lognitudinal section view of the product in FIG. 1;

FIG. 3 is a transverse section view of the product in FIG. 1;

FIG. 4 is an front view of the product in FIG. 1;

FIG. 5 is back view of the product in FIG. 1;

FIG. 6 is another side view of the product in FIG. 1;

FIG. 7 is a top view of the product in FIG. 1;

FIG. 8 is a bottom view of the product in FIG. 1;

FIG. 9 is an perspective view of the gallus buckle.

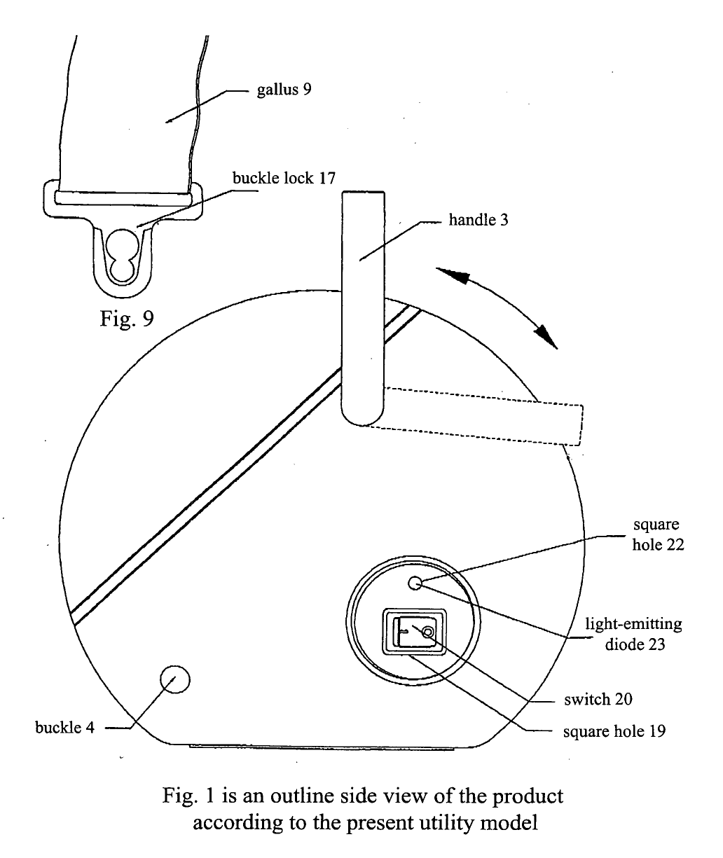

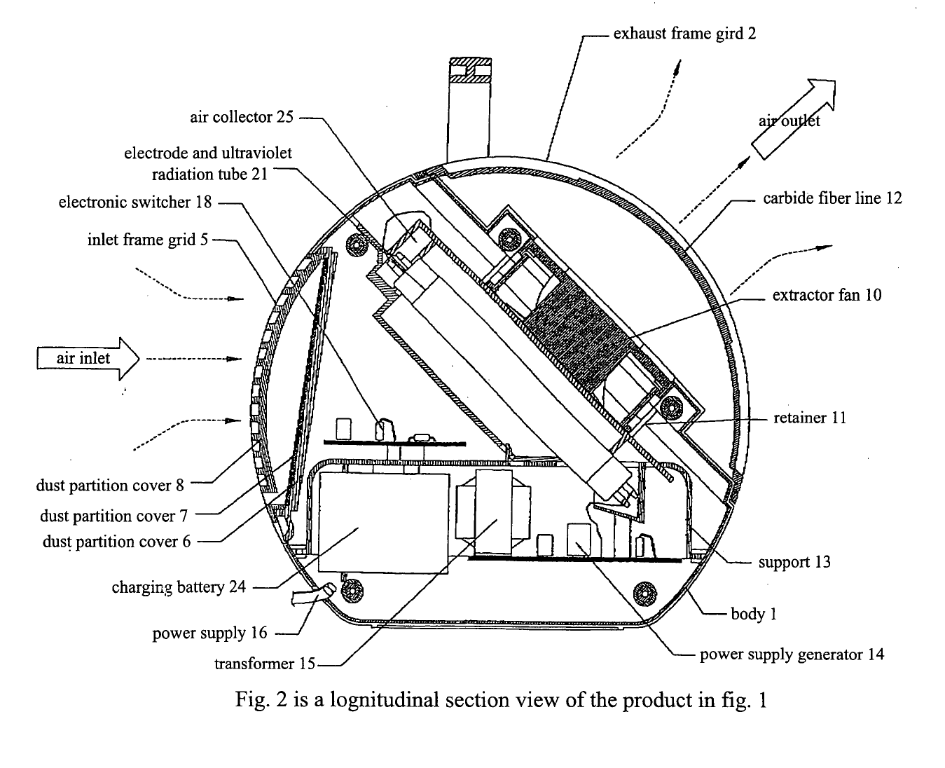

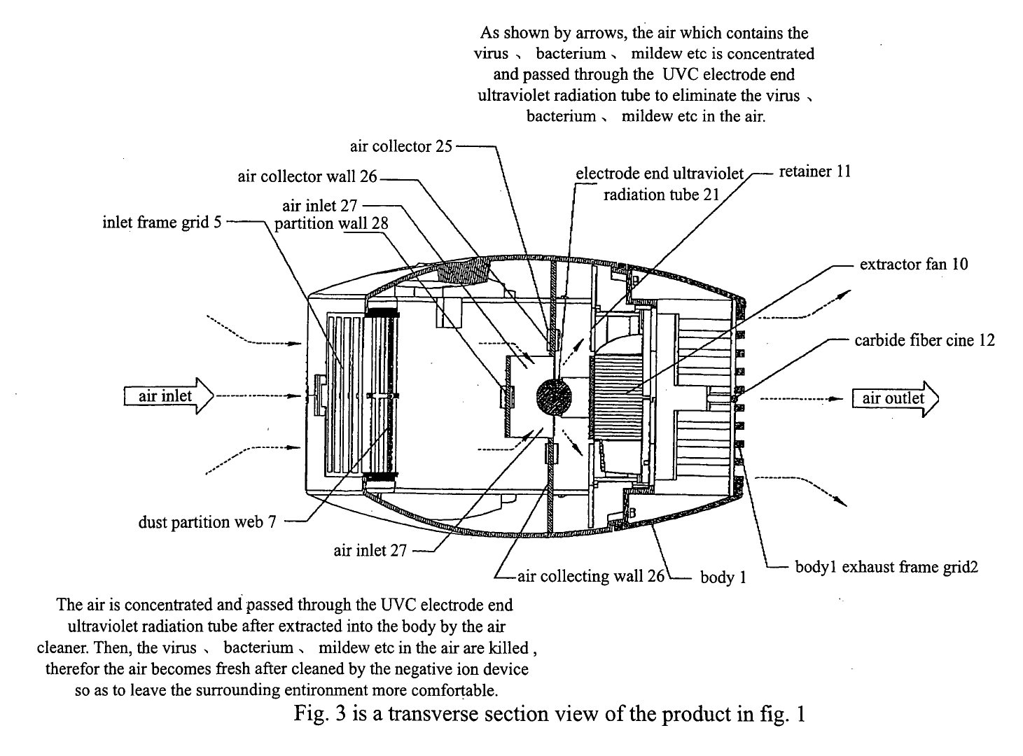

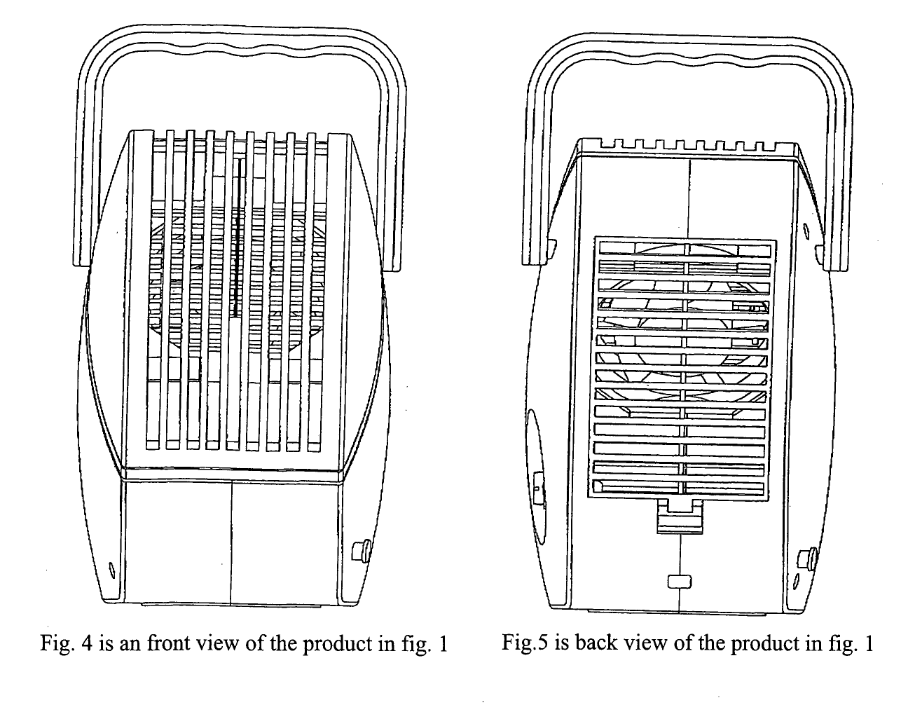

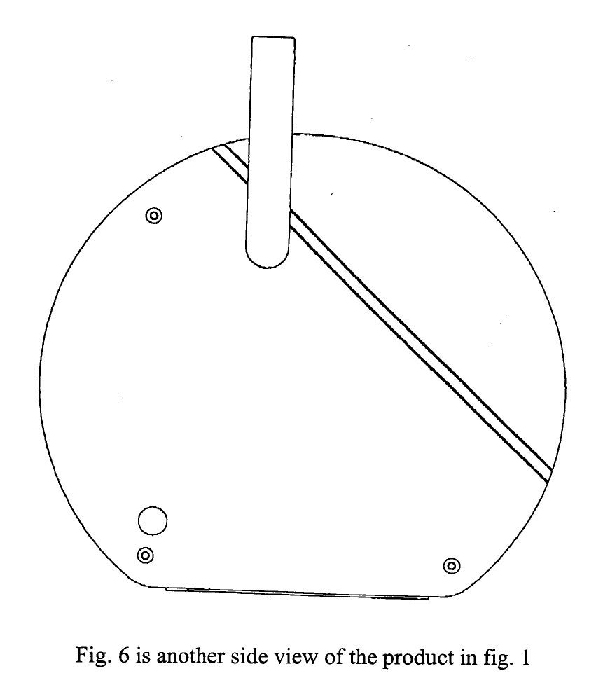

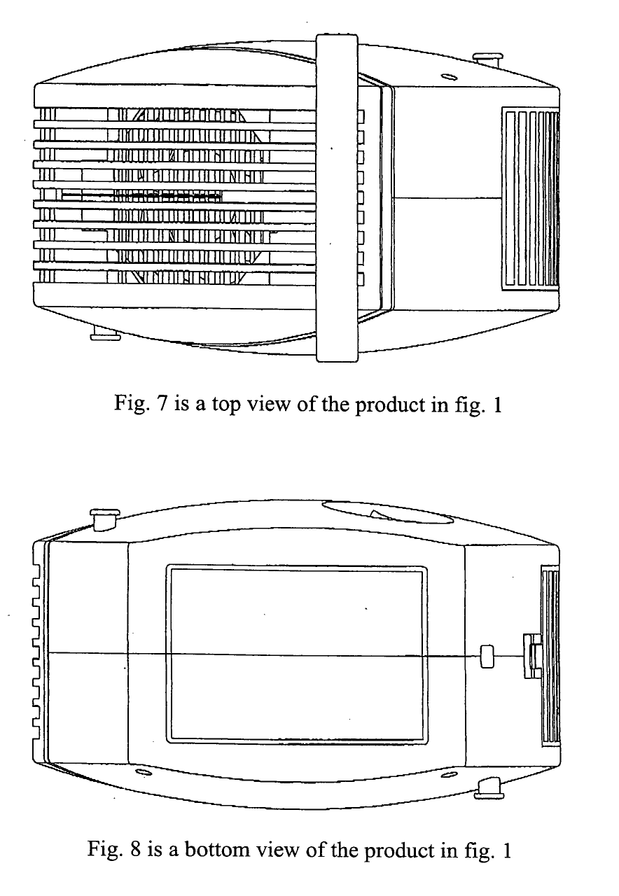

### DETAILED DESCRIPTION

Hereafter, the technical solution of the utility model will be furthermore described by referring to the embodiments in the drawings.

The Portable photoelectric air cleaner emits electrode end ultraviolet radiation rays by electrode end ultraviolet radiation tube to eliminate the virus, bacterium, mildew etc in the air which are passing the electrode end ultraviolet radiation tube. And it can increase the negative ions in the air by operating the negative ion device.

The portable photoelectric air cleaner according to the utility model mainly includes: an extractor fan, a electrode end ultraviolet radiation tube and a cathode high voltage discharge fibre line. When the air is extracted into the portable photoelectric air cleaner through the extractor fan, there will be two continuous and alternative operating modes. The first is that an outputted negative high voltage discharges intermittently (about 3 to 4 minutes) via carbide fibre so as to ionize the air to generate negative ion and exhaust it via the fan. The second is that the electrode end ultraviolet radiation tube is ignited intermittently (about 7 to 10 minutes) to emit electrode end ultraviolet radiation rays so as to eliminate the virus, bacterium, mildew etc in the air which are passing the tube. An air inlet and an outlet are formed on the portable photoelectric air cleaner. The cathode fibre line for discharging high voltage is fixed at the air outlet. The electrode end ultraviolet radiation tube is fixed nearly the air outlet which is placed within the body, and the fan is fixed nearly the air inlet which is placed within the body. Therefor, When the air which contains the virus, bacterium, mildew is drawn out through the inlet to enter the body, the moving air will easily enter and pass though the inlet grid bracket of the portable photoelectric air cleaner to arrive the outlet grid bracket thereof. Therefor, the air which contains the virus, bacterium, mildew must be contact with the electrode end ultraviolet radiation tube, thereby the electrode end ultraviolet radiation rays can eliminate the virus, bacterium, mildew in the air. Then, the air which contains negative ion and becomes pure and fresh exhausts from outlet after it is purified by the negative ion device to improve the quality of the indoor air.

Some charging batteries which can be continuously used for five to six hours is mounted in the body of the Portable photoelectric air cleaner. Therefor, it is convenient for users to carry the cleaner and breathe the pure and fresh air which has be eliminated the virus, bacterium, mildew etc, and it is effective to prevent respiratory tract infection.

The portable photoelectric air cleaner according to the present utility model furthermore includes a body which has a shape of semi-cylinder, an exhaust frame grid 2 which is formed on the front plane of the body 1, wherein the ionized air is exhausted through the exhaust frame grid 2.

FIGS. 2 and 3 show the detailed structure of the portable photoelectric air cleaner. An inlet frame grid 5 is formed on the back end of the body 1, and it provides with an air inlet used for fixing dust partition frame 6, a dust partition web 7 and dust partition cover 8. An extractor fan 10 and a retainer 11 are closed to the inner surface of the exhaust frame grid 10. A carbide fibre line 12 is formed in the center of the front surface of the exhaust frame grid 10, and it has a function of ionizing air under high voltage in the cathode.

A support 13 is provided to fix power supply generator 14, transformer 15, power supply line 16 and charging battery 24. An electronic switcher 18 is formed on the upper end of the support 13. The support 13 can protect power supply generator 14 so as to prevent current of high voltage from flowing out.

The side surface of the body 1 is formed with a square hole 19 for fixing a switch 20 which has a function of controlling the extractor fan 10, the electrode end ultraviolet radiation tube 21 and carbide fibre line 12. On the upper plane of the switch 20, there are two holes 22 for fixing light-emitting diode 23 of different color to be used for indicating.

The body 1 is connected to a movable handle 3, and the body 1 has a buckle 4 on each lower side thereof and a buckle lock 17 which can be connected to the both ends of a gallus.

In use, when the portable photoelectric air cleaner is driven, the extractor fan 10 extracts the air which contains the virus bacterium and mildew to the air collecting wall 25 in the body 1 via the air dust partition web 7 so as to easily make the air move into exhaust frame grid 2 after passing through the air collecting walls 25. The electrode end ultraviolet radiation tube 21 is formed in the center of the air collecting wall 25, that is, when the air which contains the virus, bacterium and mildew moves into the air collecting walls 25 and 26, and then moves into the air inlet 27 placed between the electrode end ultraviolet radiation tube 21 and the air collecting wall 26, the electrode end ultraviolet radiation tube 21 will emit the electrode end ultraviolet radiation rays to eliminate the virus, bacterium and mildew in the air. Meanwhile, the cleaned air is exhausted to the negative high voltage carbide fibre line 12 of the exhaust frame grid 2 so as to generate negative ions. The pure and fresh air which contains negative ions is exhausted by the exhaust frame grid 2, thereby the quality of the indoor can be improved.

Both the front and back side of the air collecting wall 25 are provided with the partition wall 28 for preventing the electrode end ultraviolet radiation rays from transmitting out the body 1 so as to protect users' eyes.

The circuit principle of the portable photoelectric air cleaner is that: the power supply is inputted to the negative ion high voltage generating circuit via a trigger 20 to output a negative high voltage. The power supply is also provided to an extractor fan 10 via a full-wave rectifier and a velocity controlling circuit so as to drive the electrode end ultraviolet radiation tube 21 and the full-wave rectifier via direct current-alternating current converter. On the other hand, the power supply is provided to the automatic circular controller which belongs to the activating circuits of the negative ion generator and the electrode end ultraviolet radiation tube 21 via a direct current stabilizer. Thereby it can control the negative ion generator and the electrode end ultraviolet radiation tube 21 to operate alternatively. The preferred configuration of the circuit has been disclosed in the other related application of the present inventor and is the prior art, it is omitted here. The charging battery is mounted in the body of the portable photoelectric air cleaner of the present utility model, it can be continuously used for five to six hours. Therefor, the user can breathe the fresh and clean air which has been eliminated the virus, bacterium mildews etc in deferent places.

The portable photoelectric air cleaner and the electrode end ultraviolet radiation tube 21 of the present invention can generate the electrode end ultraviolet radiation rays which have a wavelength of 253.7 nm. It is proved after scientific research that the ultraviolet with such wavelength can effectively kill the virus, bacterium, mildew etc in the air. Furthermore, the increase of negative ions in the air by operating the negative ion device can promote biochemical effect and decrease the secretion of hormone which makes someone depressed and tired. In modern world, in most of families, hospitals, department stores, cinemas, restaurants, offices, workshops, elevators, every type of vehicles ships planes and trains, the portable photoelectric air cleaner is able to make the environment of people's life, dwelling, treatment, working, consumption, getting on a vehicle and so on improved, thereby it gives people a pure space in the modern world to which the environment is getting to be destroyed.

The present invention has been described with regard to the technical solutions and embodiments. However, it is not intended to limit the technical solutions of the application. It is obviously that there are a variety of forms to obtain the utility patent, and the person skilled in the art will make various modifications according to the sprits and ranges of the claims.

1. A portable photoelectric air cleaner, which includes a semi-circular body in which there are an extractor fan a transformer a circuit board an electrode end ultraviolet radiation tube and a cathode high voltage discharge fibre line, which is characterized in that an air inlet and an outlet are formed on said body. The cathode fibre line for discharging high voltage is fixed at the air outlet. The electrode end ultraviolet radiation tube is fixed at the outlet which is placed in the body, and is formed in an air collector, the extractor fan is fixed between the electrode end ultraviolet radiation tube and the air outlet which are formed nearly inside the body.

2. The portable photoelectric air cleaner according to claim 1, the furthermore character is that said air collector is comprised of a space which is defined by an air collecting wall and blocking wall, the electrode end ultraviolet radiation tube is fixed between the front and back block walls so that the rays can't emit out of the cleaner.

3. The portable photoelectric air cleaner according to claim 1, wherein a supporting frame is formed in said body, the lower end of the supporting frame is provided with a power supply generator a transformer power source lines and a charging battery, and the upper of the same is provided with electronic switcher.

4. The portable photoelectric air cleaner according to claim 1, wherein a movable handle and a movable gallus are fixed on the body, said movable gallus is connected to the body though a buckle.

5. The portable photoelectric air cleaner according to claim 1, wherein the body has charging batteries for providing power to the air cleaner, the output of said charging batteries is connected to the direct current(DC) inputs of a circuit board.

6. The portable photoelectric air cleaner according to claim 2, wherein the body has charging batteries for providing power to the air cleaner, the output of said charging batteries is connected to the direct current(DC) inputs of a circuit board.

7. The portable photoelectric air cleaner according to claim 3, wherein the body has charging batteries for providing power to the air cleaner, the output of said charging batteries is connected to the direct current(DC) inputs of a circuit board.

8. The portable photoelectric air cleaner according to claim 4, wherein the body has charging batteries for providing power to the air cleaner, the output of said charging batteries is connected to the direct current(DC) inputs of a circuit board.
